(19) United States
(12) Patent Application Publication
(10) Pub. No.: US 2009/0227930 A1
(43) Pub. Date: Sep. 10, 2009

(54) Hygiene materials and absorbents

(76) Inventor: William E. Crisp, Paradise Valley, AZ (US)

Correspondence Address: Barbara E. Johnson, Esq., 555 Grant Street, Suite 323, Pittsburg, PA, 15219, US

(21) Appl. No.: 12/380,111

(22) Filed: Feb. 23, 2009

Related U.S. Application Data
(63) Continuation-in-part of application No. 11/130,800, filed on May 17, 2005, now Pat. No. 7,495,146.
Application No. 11/130,800 is a continuation-in-part of application No. 10/891,566, filed on Jul. 15, 2004, now abandoned.

(51) Int. Cl. A61F 13/53; A61M 35/00; A61F 11/08; A61L 15/44; A61K 9/02; A61F 7/08; A61K 9/06

(52) U.S. Cl. 602/48; 604/367; 604/289; 128/858; 128/864; 604/288; 607/112; 604/93.01

(57) ABSTRACT

A topical product including but not limited to a feminine hygiene product or incontinence shield which contains at least two kinds of particulate metals distributed throughout which, when in contact with moisture, form a galvanic current (virtually if not completely imperceptible to the user) that has antimicrobial effect on all pathogens. Galvanic current virtually or completely imperceptible to the user facilitates antimicrobial effect.

### CROSS REFERENCE TO RELATED APPLICATIONS

This patent application is a continuation-in-part of U.S. application Ser. No. 11/130,800, filed May 17, 2005, which was a continuation-in-part of U.S. application Ser. No. 10/891,566, filed Jul. 15, 2004, each of which is hereby incorporated herein by reference.

### BACKGROUND OF THE INVENTION

1. Field of the Invention

The present invention relates to an apparatus for attacking microbes, namely, bacteria, viruses and fungi. More particularly, the present invention is a sustained release microvoltage device for use as any sort of topical or mucosal hygiene material or absorbent, including but not limited to feminine hygiene products, incontinence shield products for either sex, comfort pads of various types, test materials and topical or mucosal gels, lotions and lubricants.

2. Orientation to the Prior Art

At this writing, polymer absorbents are largely regarded with skepticism for use in feminine hygiene products. A few decades ago, certain tampons containing certain polymer absorbents or other ingredients were removed from the market, or in some cases provided with a warning label, after questions arose regarding the tampons' possibly contributing to increased incidence of Toxic Shock Syndrome. These and other events led to a state of the art, today, that in many cases favors cellulose- and cotton-based absorbents for feminine hygiene products. The performance of cellulose and cotton-based absorbents is not optimal, however, and certain products today already have certain enhanced absorbency materials besides cellulose or cotton to improve function. Also, some manufacturers contend that non-organically produced cotton is among the most pesticide-laden material grown modern agriculture, which if true makes non-organic cotton a poor choice for use in feminine hygiene products. Also, manufacturing processes such as the bleaching of cotton or cellulose are believed to create certain unwanted chemicals, such as dioxins, which if they come in contact with the skin or mucosa of a feminine hygiene user then create separate wellness or disease risk.

Alongside the above challenges, consumers of feminine hygiene products continue to need improvements in easy self-diagnosis of conditions such as sexually transmitted diseases (such as Chlamydia, Gonorrhea and Syphilis) or infections with Trichomonas or Monilia (Candida) or even self-exams for indications or pre-indications of cervical cancer such as are usually assessed by Papanicoulaou (“Pap”) test diagnostics.

Even beyond the challenges in the feminine hygiene manufacturing industry, other topical epidermal and mucosal products are at this writing lacking in antimicrobial or bacteriostatic features because, as a general rule, materials applied topically to the skin or mucosa inevitably create an optimal environment for microbial growth. All kinds of topical materials, therefore, need improvement as to antimicrobial properties, including but not limited to incontinence shields, joint pads, adhesive heating pads, rectal comfort pads, earplugs, and even gels lotions and creams intended for topical epidermal and mucosal application. Anywhere that skin or mucosa are covered with any sort of conforming polymer or polymer-containing article or construct, a need for improved antimicrobial action is still needed at this writing. Accordingly, a need remains for an antimicrobial technology which can enhance the performance and safety of topical epidermal and mucosal constructs and materials including but not limited to feminine hygiene articles, joint pads, adhesive heating pads, incontinence pads and shields, rectal comfort pads, suppositories, earplugs, nose plugs, and topical gels lotions and creams.

### SUMMARY OF THE INVENTION

In order to meet this need, the present invention is any polymer-containing topical epidermal or mucosal article or material which contains, as described herein, at least two kinds of particulate metals distributed throughout which, when in contact with moisture, form a mild galvanic current (virtually if not completely imperceptible to the user) having an anti-microbial effect. Throughout the below specification, for simplicity the preferred embodiment metal particles of silver and zinc are often referred to specifically, but the invention involves the use of any two metals which, in dispersed in a polymer-containing article or material in particulate form, can create a mild galvanic current when exposed to body fluids. The articles or materials can be any topical epidermal or mucosal construct including but not limited to feminine hygiene articles and their adjunct diagnostic tools; incontinence shields, joint pads, adhesive heating pads, rectal comfort pads, suppositories, earplugs, noseplugs and topical and mucosal gels, lotions and creams.

### BRIEF DESCRIPTION OF THE FIGURES

FIG. 1 is a perspective view of a tampon containing particles of two metals as described further below;

FIG. 2 is a vaginal probe (swab) for testing pH of vaginal fluids.

FIG. 3 is a perspective view of a standard sanitary napkin containing particles of two metals as described further below.

FIG. 4 is a plan view of a sanitary napkin “with wings,” containing particles of two metals as described further below.

FIG. 5 is a perspective view of an incontinence shield (adult diaper) according to the present invention, containing particles of two metals in the central absorbent material thereof.

### DESCRIPTION OF THE PREFERRED EMBODIMENTS

The present invention is any polymer-containing topical epidermal or mucosal article or material which, because it contains at least two metals in particulate form that create a mild galvanic current when exposed to moisture, has anti-microbial characteristics. In order to accomplish the creation of a mild galvanic current, the first metal must have an electrochemical potential and the second metal must have an electrochemical potential different from the first metal (i.e., having different valences) and be arranged or arrayed adjacent one another. Typically and preferably the two metals are silver and zinc but other metals are disclosed below and embraced by the present invention. The anti-microbial characteristics created by the mild galvanic current in turn allow for the incorporation of a wider variety of absorbent materials—such as synthetic polymers—in articles such as feminine hygiene products than may at this writing be acceptable for use. The galvanic current provides not only health benefits in the mucosal or other areas, but reduces proliferation of pathogens that may contribute to Toxic Shock Syndrome or other wellness or disease risks. The galvanic current directs pathogens to the positively charged anode where they are killed, because all pathogens are negatively charged. In an especially preferred embodiment of the invention, an absorbent gel polymer is incorporated in the feminine hygiene product or incontinence shield so that the absorbent gel polymer conforms closely to the surface of the adjacent anatomy. In particular, a tampon according to the present invention will preferably be manufactured to contain enough absorbent gel polymer that the tampon, generally cylindrical before insertion into the vagina, will largely flatten to the generally flattened normal configuration of the vagina while also conforming with the natural rugae of the vagina, to eliminate interstices within which pathogens might otherwise hide. The tampon of the present invention may thus be used for antimicrobial action in the vagina even when menses are not occurring—or even in post-menopausal or postpartum women.

By “particulate” is meant a form of each metal other than a substantial or solid sheet, so that a dispersed or intertwined collection or matrix of both (at least two or more) metals will create a mild galvanic current upon exposure to electrolytic fluid or moisture. “Particulate” therefore embraces particles having diameters between about 0.1 to 1.0 microns or any other shape of metal including but not limited to threads, cords, shards, fibers or flecks in which one dimension of the shape falls within the 0.1 to 1.0 micron range. The metal particles of the invention may not be visible to the naked eye. The particles are generally spaced apart between 0.1 and 7 mm, more preferably from 2.0 mm to touching, to create a microcurrent. The particles either touch one another or act independently to kill pathogens.

While it is known that the use of a low voltage field applied through a reservoir can be used to deliver drugs or agents in the reservoir systematically or to produce a localized therapeutic effect, it is believed that the use of metals to enhance a feminine hygiene product (or any other topical epidermal or mucosal product other than a wound dressing) as described particularly herein has not been developed before. Mild current is also known for use in controlling pain, edema and acceleration of wound healing. Moreover, when one or more vaginal topical medicaments are administered while the feminine hygiene article is adjacent, the localized effect of the medicament and its absorption through the mucosa will be greater than when the topical medicament is administered alone. For this reason, the use of feminine hygiene products according to the invention with other topical medicaments is believed to enhance the delivery of those medicaments when present.

One of the metals best suited for use in particulate form, for distribution through the feminine hygiene product of the invention as described below, is silver. Silver has been used as a disinfectant for centuries. The use of medicinal silver was for a while diminished by the advent of more versatile and effective antibiotics. However, the misuse of antibiotics coupled with bacterias' ability to mutate have resulted in resistant organisms and reawakened interest in silver's effective antimicrobial properties in general. Elemental silver is an effective microbicide in solutions as dilute as one part per 100 million. Silver ions kill microorganisms by (it is believed—see below) blocking the respiratory system, which is the process of harvesting energy by transferring electrons from an electron donor to an electron receptor.

When silver is used in the present invention, the silver particles are preferably silver that is at least 99.99% to 99.9999% pure. However, less pure silver, and suitable salts and oxides thereof may be used. Examples of other silver particles that may be used include: silver fluorides, silver chlorides, silver bromides, silver iodides, silver oxides, silver sulfides, silver selenides, and silver tellurides. Preferably, the diameter of the silver particles is between 0.1 to 1.0 microns, however, the diameter may vary.

The other metal particles of the present invention are preferably zinc, however other metal particles that will create a galvanic charge when adjacent to silver may be used, or any two metals with different electrochemical potentials may be used. The other metal particles besides silver are desirably pure or nearly pure zinc, but aluminum, cobalt, copper, gold, iron, magnesium, platinum and titanium may also be used. In addition, the other metal particles may comprise compounds providing free dissolution of metal ions, such as zinc gluconate, zinc acetate, zinc chloride, zinc citrate, zinc propionate, zinc sulphate heptahydrate, zinc butyrate, zinc formate, zinc glycolate, zinc glycerate, zinc lactate, zinc sulfate, ferrous oxide, ferrous sulphate, and titanium oxide. Other zinc salts which are soluble in water and have low pK values, which indicate a high rate of zinc ion release, may also be used. Other suitable metal salts and compounds that release metal ions upon exposure to an aqueous medium are likewise suitable. Preferably, the diameter of the other metal particles is between 0.1 to 1.0 microns, that is, the same as the diameter of the silver or first-described particles; however, the diameter may vary.

A galvanic current of approximately 0.2 millivolts will form in any adjacent physiological fluids (such as menstrual fluids) and thus for example throughout the genital mucosal surface during use of the present feminine hygiene product during menstruation. The galvanic current may range from about 0.1 to 1.0 millivolts. The galvanic current and concomitant anode act as an antimicrobial against bacteria, viruses, and fungi. Without intending to be bound by the theory, the antimicrobial action is believed to occur as follows: all bacteria, viruses, and fungi, because they are negatively charged, are drawn by the galvanic current to the anode, and the anode causes the organism to adhere to it, with the combination of the current's and the adherence' interfering with the function of the sulfhydryl (SH) groups of the microbes. This in turn disrupts the respiratory pathway of the organisms which kills the organisms. Along with their antimicrobial properties, the silver particles draw edema or fluid from the mucosal area to decrease swelling which increases the capillary blood flow, which in turn promotes mucosal health. Furthermore, ions from the other metal particles provide, in the case of zinc in particular, therapeutic benefits including but not limited to, control of viruses and autolytic debridement of any wounds or scar tissue present. In the preferred embodiment, zinc is used because zinc is necessary for a wide variety of metabolic processes, including the synthesis as well as the degradation of nucleic acids, proteins, carbohydrates, and lipids, and thus provides an apt topical or transdermal essential metal in addition to its galvanic-current-creating effect.

An exemplary absorbent polymer throughout which the metals of the present invention may be dispersed is composed of two elements, a hydrophilic, or water-loving, polymer and a fibrous material such as wood pulp. The polymer is typically made of fine particles of an acrylic acid derivative, such as sodium acrylate, potassium acrylate, or an alkyl acrylate. These polymeric particles act as tiny sponges that retain many times their weight in water. Microscopically these polymer molecules resemble long chains or ropes. Portions of these chemical “ropes” are designed to interact with water molecules. Other parts of the polymer have the ability to link chemically with different polymer molecules in a process known as cross linking. When a large number of these polymeric chains are cross linked, they form a gel that is both water insoluble and hydrophilic. Polymers with this ability are referred to as hydrogels, superabsorbents, or hydrocolloids. Depending on the degree of cross linking, the strength of the gel can be varied. This is an important property because gel strength is related to the tendency of the polymer to deform or flow under stress. If the strength is too high the polymer will not retain enough water. If it too low the polymer will deform too easily, and the outermost particles in the pad will absorb water too quickly, forming a gel that blocks water from reaching the inner pad particles. This problem, known as gel blocking, can be overcome by dispersing wood pulp fibers throughout the polymer matrix. These wood fibers act as thousands of tiny straws which suck up water faster and disperse it through the matrix more efficiently to avoid gel blocking and, in the context of the invention, to promote conformation of the polymer to the desired anatomic area. Manufacturers of disposable diapers, which use such absorbents, have to date optimized the combinations of polymer and fibrous material to yield the most efficient absorbency possible and the preparation of these absorbent polymer matrices is well known at this writing. Such optimization can be used in the preparation of feminine hygiene products of the present invention (or incontinence shields—see below). The metal particles referred to above can be dispersed throughout the article or throughout a portion of the article that will come in contact with the skin, mucosa or other anatomic surface.

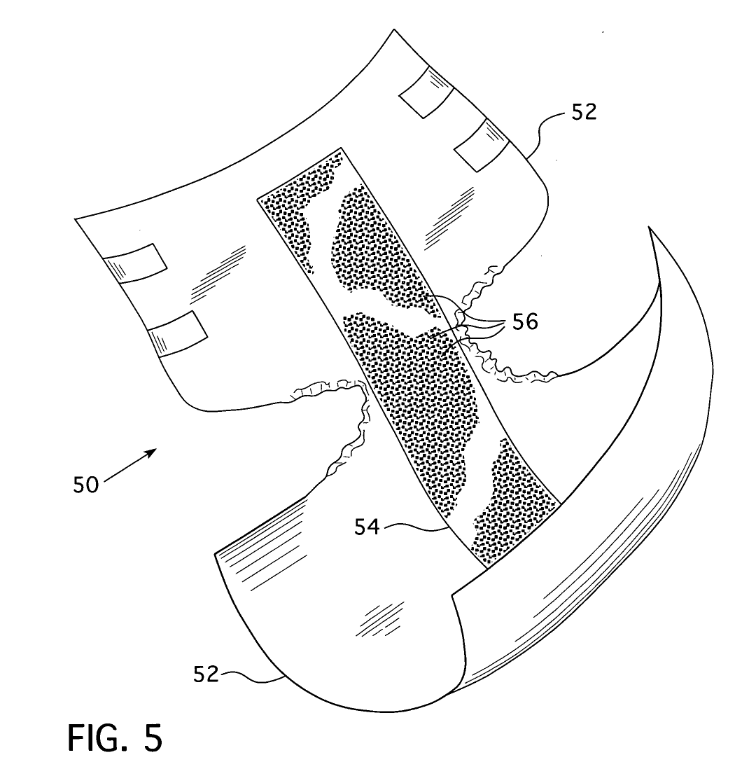

The present technology is equally applicable to incontinence shields such as are used by people experiencing urinary incontinence, usually but not always correlating with advancing age. The manufacture of incontinence shields with the two metals of the present invention means that when the incontinence shields become wet, during use, the metals create a galvanic current which in turn helps to reduce or eliminate microbes on the adjacent skin surfaces. Not only does this improvement to incontinence shields benefit the patient to reduce the concerns of odor and accidental failure, the antimicrobial effect is beneficial in many ways including but not limited to reduction of decubitus ulceration in those for whom movement is impaired. While there is no substitute for the frequent changing of an incontinence shield for a mobility-impaired patient, the concomitant reduction in microbes on the skin is a quantifiable benefit in elder care. A typical incontinence shield is shown in FIG. 5, discussed further below.

Other forms of the present invention besides feminine hygiene articles and incontinence shields include, without limitation, earplugs, joint pads, nose plugs, adhesive heating pads, rectal comfort pads, suppositories, and topical epidermal or mucosal gels, lotions and creams including but not limited to vaginal gels and nasal gels.

Returning now to the feminine hygiene application, there are three basic types of microbe-related vaginitis which the present invention can ameliorate or eliminate. Yeast vulvovaginitis is usually caused by Candida, which is a type of fungus commonly referred to as yeast. Although it is usually found on the skin and in the gastrointestinal tract and vagina, on occasion and during overgrowth it can invade the tissues of the vagina and cause severe inflammation. This type of yeast vaginal infection usually causes itching and a thick white discharge. Sometimes the only symptoms are external or internal burning or pruritis, especially in diabetics and AIDS patients. Yeast infections are extremely common and cause much unmentioned suffering, and a feminine hygiene product which can reduce or eliminate the incidence and discomfort is a much-needed consumer product at this writing. Second, bacterial vaginosis occurs when the normal, “good” bacteria of the vagina, Lactobacillus, are overrun by “bad” bacteria. Finally, trichomonas is a one-celled organism that can cause a sexually transmitted infection. In men, the infection only lasts a few weeks and often is not associated with any symptoms. However, women can harbor the infection for years. Some will have accompanying symptoms of discharge and irritation, while others may be symptom free. Sometimes the diagnosis of Trichomonas infection is made only after the organism appears in a Pap test. The feminine hygiene products of the present invention can reduce or eliminate all of fungal, bacterial or Trichomonal irritation or the sexually transmitted diseases including Chlamydia, Gonorrhea and Syphilis, any one of which can cause infertility, cervicitis or endometriosis. The galvanic current is mild enough that the predominant bacteria of the vagina, Lactobacillus, are generally not completely or even predominantly eradicated and can quickly return to normal population levels after use of the feminine hygiene product ceases. In fact, when the galvanic current is created in the vagina together with a reduction in pH (see below) growth of Lactobacillus will actually be encouraged in favor of pathogens that will not thrive in the acidified environment.

That Trichomonas is sometimes diagnosed by a Pap test sets the stage for an understanding of an adjunct feature and benefit of the present invention—it can be arranged as a collector of cells not only for diagnosis of cervical cancer if present, but for diagnosis of any microbial infection that may be present. Genital herpes is also a known causative factor in cervical cancer and vaginal cancer.

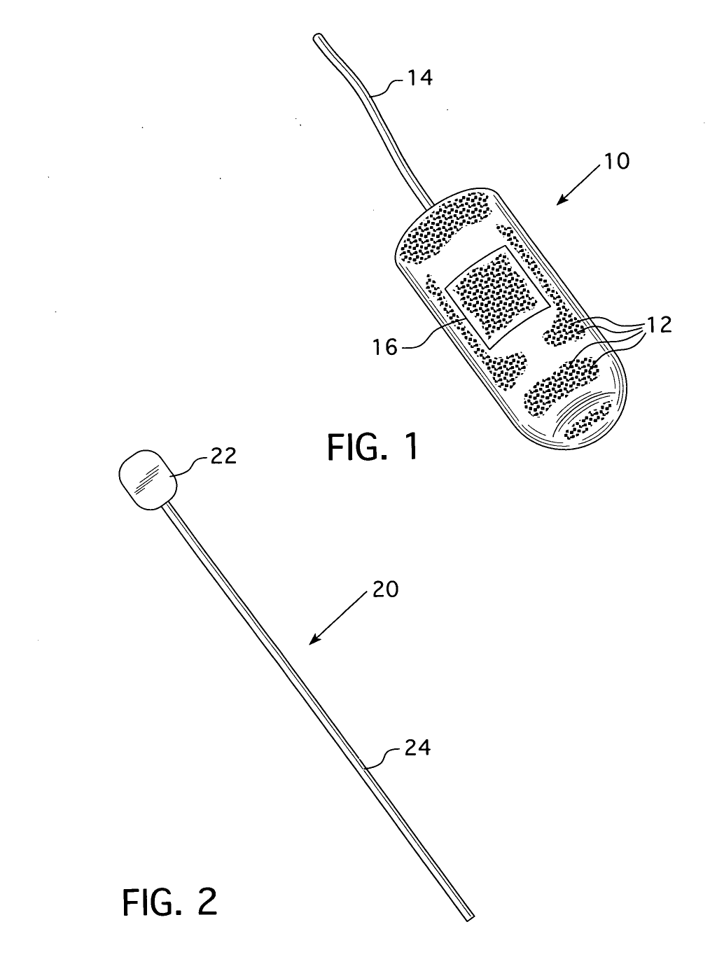

Referring now to FIG. 1, the invention embraces a tampon 10 in which the metals of the invention have been dispersed throughout. The tampon shown in perspective view in FIG. 1 contains two types of metal particles 12 (shown larger than actual size) having different electrochemical potentials. The particles 12 are of two different metals and the particles 12 can be of the same or of different sizes. The tampon 10 generally contains a quantity of absorbent material of any suitable kind covered by an optional outer mesh (not shown) and attached to a pull string 14. A square guideline (marking) 16 on the tampon guides the user to place the denoted area over the cervix during use, so that the area which contacted the cervix can be used for a post-use smear onto a slide or other diagnostic substrate, for the user to send directly to a laboratory for analysis without need for a physician to take a Pap test smear by swab. If desired, the guideline area may be provided with a slightly more porous and/or abrasive surface to assist in the sampling and retention of cervix cells both while the tampon is in use and during its removal.

As an adjunct to any of the present products, a pH test may be used to assess the health of the vaginal area. It is known at this writing that certain vaginal pH range indicates monilia or candida overgrowth, that another pH range can indicate Trichomonas, and that a third pH range can identify one or more possible veneral disease states. As an adjunct to the use of the present products, therefore, a pH indicator (containing pH paper known in the art) may be placed on the tampon itself or, according to FIG. 2, on a stick or swab to be used as a probe. The probe 20 of FIG. 2 includes a test surface 22 and a handle 24. The test surface 24 includes, on either or both sides, pH indication chemicals and carriers therefor known in the art. Although the diagnostic ranges of vaginal pH do not form a part of the present invention, because they are known at this writing and vary for different conditions and microbes, the joint use of pH assessment together with a feminine hygiene product containing galvanic-current-generating metals forms a part of the invention. These pH indicators are not conventional pH papers but instead represent new, more sensitive and specifically calibrated and color coded pH strips or paper that identify individual pathogens according to these known pH ranges.

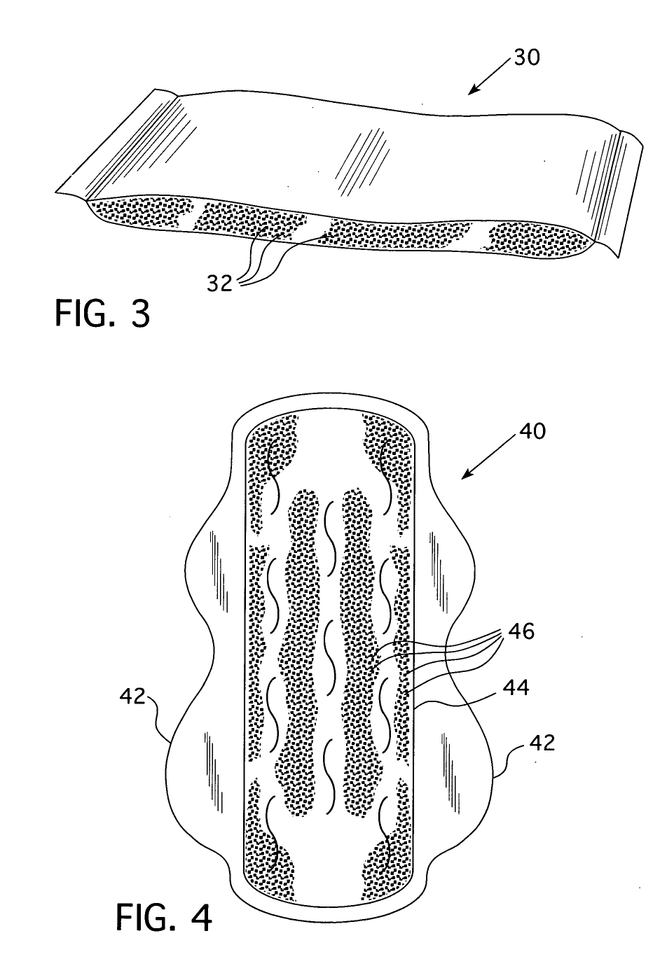

Referring now to FIG. 3, a sanitary napkin containing two types of metals dispersed throughout is shown in perspective view. Metal particles 32 are shown larger than actual size and include at least two different metal types to create the anode and cathode described elsewhere herein. The particle sizes may be the same or different within the size ranges disclosed in this specification.

Referring now to FIG. 4, a feminine hygiene pad 40 having adhesive wings 42 known in the art is shown in plan view, with the central pad 44 containing dispersed metal particles 46. Metal particles are shown larger than actual size and may vary in size.

FIG. 5 illustrates one embodiment of an incontinence shield 50 according to the invention, in which a shaped barrier 52 has on the interior surface thereof an absorbent area 54 containing dispersed metal particles 56. The metal particles are shown larger than actual size and may vary in size.

Polymer materials which may be included in any the above-identified products include, without limitation, polyurethane polymers, polyester polymers, polyethylene, polypropylene and polybutylene polymers, acetic acid polymers, acrylic and acrylate polymers, polycarbonate and other synthetic polymers. In addition a wide variety of absorbent polymers may be incorporated into the feminine hygiene or incontinence product including, without limitation, polysaccharides such as cellulose or carbocellulose based materials, alginates, and other known polysaccharide absorbents.

In wound dressings, the metals which produce the galvanic current need to be encapsulated at least partially in erodible materials to create a slow exposure of the metal particle surface to the electrolyte fluid to create a sustained-release galvanic current. Surprisingly, the present disclosed materials, intended for intact or largely intact epidermis or mucosa, do not require such erodible materials, and the inventive constructs work best when the particles of the two metals are dispersed throughout the material without additional provision for controlled release. For the feminine hygiene articles, therefore, in theory, without intention of being bound by the theory, the very slow flow or intermittent flow of menstrual fluid or urine (or other body fluids exclusive of wound settings) is gradual enough that the slow or intermittent flow itself provides the necessary controlled release of the metals' exposure and resulting galvanic current upon exposure to electrolyte. For the other articles and materials disclosed herein a similar dynamic is believed to apply—the intact mucosa or epidermis and its natural moisture provides a sustained or controlled slow contact with the dispersed metals to sustain the mild galvanic current over time.

For the disclosed constructs other than feminine hygiene and incontinence products, the same shapes and structures (or lack thereof, in the case of creams and lotions) are used as are typical in the prior art. The common features of all the disclosed articles and materials herein are that a) they are for topical epidermal or mucosal application; b) they are designed for use adjacent healthy or reasonably intact epidermis or mucosa; and c) they contain at least one polymeric constituent that contributes to a close conformation if not some level of vacuum seal between the material and the adjacent skin or mucosa. All of the materials disclosed herein—the polymer-containing feminine hygiene products, the other constructs, and the creams and lotions—all are united in that they contain at least one polymer in addition to the dispersed metals, and that the dispersed metals create a safer topical or mucosal environment considering the adjacent position of the one or more polymers and the skin or mucosal than could be achieved with prior art technology.

Any cream, lotion or gel may be used as the polymer-containing material to which the present metals are added, for further application to the skin or mucosa. Such formulations are well known at this writing and virtually any gel, lotion or cream for topical or mucosal application becomes more beneficial when the present particulate metals are incorporated. The concentration of the metals should be that which, when dispersed, results in the above-described spacing, and the particle sizes for gels, lotions and creams should be in the same range as described above.

Gel-forming constituents can include, without limitation, Polysorbate 40 (a polyhydroxy organic compound), Poloxamer 407 and Hexylene glycol, mixed with purified water according to the skill of the art. Other ingredients, again without limitation, can be ethylene diamine tetraacetic acid disodium, ascorbic acid, Carbomer 934P, Poloxamer 407, polyethylene glycol, Polysorbate 40, hexylene glycol, butylated hydroxytoluene, butylated hydroxyanisole, benzyl alcohol, polyethylene glycol (PEG) or tromethamine, all of which are known at this writing for their uses as surfactants, stabilizers, solubilizers and neutralizers in topical gels, lotions and creams.

A typical gel may contain the following concentrations of each ingredient in an exemplary but by no means limiting formulation (concentration (% W/W) of ingredients: Purified water 49.20%; Edetate Disodium Stabilizer 0.05%; Ascorbic acid Stabilizer 0.05%; Carbomer 934P Thickening 1.25%; agent Poloxamer 407 Surfactant 0.2%; PEG-400 Co-solvent 45.0%; Polysorbate 40 Surfactant 0.2%; Hexylene glycol Co-solvent 2.0%; Butylated hydroxytoluene Stabilizer 0.05%; Butylated hydroxyanisole Stabilizer 0.05%; Benzyl alcohol Preservative 1.0%; Triethanolamine Neutralizer 0.8%, balance purified inert materials. The gel is admixed according to methods well known in the art. A quantity of two metals as described above is admixed in such amount as to give a particle spacing of between 0.1 and 7 mm without the particles' touching, when the metals are fully dispersed. Because of the fine size of the particles (diameters between 0.1 and 1.0 microns) the metals tend to remain in full dispersion indefinitely and have excellent shelf life. Any gel cream or lotion having a viscosity of at least about 2000 Centipoise is suitable for use as a base cream, gel or lotion for which the present metals may be added.

A particular gel for use as a vaginal gel is the above-described gel, set forth in the immediately preceding paragraph, to which 1-15%, more preferably 2-8%, by weight of alum (hydrated aluminum potassium sulfate) has been added together with the metal particles of the present invention. The alum should ideally be sterilized culinary grade alum powder. The purpose of the addition of the alum is to increase the acidity (reduce the pH) of a gel for vaginal application when the pH of the vagina should be reduced. Traditional vaginal douches have in some decades been vinegar based, and the use of acidic vaginal treatments is not new by any means. However, the combination of alum powder and the particulate metals of the present invention is believed to give new and unexpected results in both the acidification and antimicrobial treatment of the vagina (or other mucosal surfaces in need of such treatment). In addition, the negative pH of the composition and in the vagina stimulates the growth of lactobacillus, which is the dominant bacteria of a healthy vagina, while reducing or eliminating pathogens.

The gel of the present invention, containing at least two metals in particulate form with or without additional ingredients, is particularly well suited as a gel for application to the nasal mucosa to create an antimicrobial effect there. Although at this writing a popular over-the-counter remedy is a nasal gel containing zinc, the present nasal gel contains at least two metals which, particularly when formulated with particle spacing of 2 mm to touching, create a mild galvanic current in the nasal mucosa to create a desired antimicrobial effect. Many experts believe that reducing pathogens in the nasal mucosa can shorten the symptoms and severity of the common cold.

It should be understood that the articles and compositions of matter disclosed herein are very similar to the wound dressing materials disclosed in the parent specifications identified above, except that particular provision of controlled-release technology in articles and compositions generally intended for topical application to intact or largely intact epidermal or mucosal surfaces is not as necessary as it is in a wound dressing context. However, shields and pads according to the present invention may in fact be used on wounds when a wound dressing according to U.S. application Ser. No. 11/130,800 is unavailable for any reason, because the present constructs and materials are well suited to all epidermal and mucosal applications for antimicrobial action.

Although the invention has been described above with particular reference only to certain materials and methods, and constituents, the invention is only to be limited insofar as is set forth in the accompanying claims.

The invention claimed is:

1. A feminine hygiene product comprising at least one polymer and at least two types of metals in particulate form distributed throughout said at least one polymer.

2. The feminine hygiene product according to claim 1, wherein the metal types are silver and zinc.

3. The feminine hygiene product according to claim 2 wherein the particles have diameters between 0.1 and 1.0 microns.

4. The feminine hygiene product according to claim 3 wherein the particles are spaced between 0.1 and 7 mm apart or are touching.

5. The feminine hygiene product according to claim 4 wherein the particles are spaced 2.0 mm or less apart or are touching.

6. An incontinence shield for use by a patient experiencing potential urinary. incontinence, comprising a quantity of absorbent material and at least two types of metals, in particulate form, distributed throughout said absorbent material.

7. The incontinence shield of claim 6 wherein the metal types are silver and zinc.

8. The incontinence shield of claim 7 wherein the metal particles are spaced between 0.1 and 7 mm apart without touching.

9. A topical absorbent product for feminine hygiene or incontinence shield use, comprising at least one absorbent layer including a plurality of first metal particles having an electrochemical potential, a plurality of second metal particles having a different electrochemical potential from said plurality of first metal particles, all of said metal particles' being dispersed throughout said absorbent layer, said metal particles being spaced from between 0.1 mm to 7.0 mm apart or are touching and which due to their spacing generate a sustained galvanic current of between 0.1 to 1.0 millivolts when subjected to an electrolyte-containing fluid.

10. A topical absorbent product for feminine hygiene or incontinence shield use, comprising at least one absorbent layer including a plurality of first metal particles having an electrochemical potential, a plurality of second metal particles having a different electrochemical potential from said plurality of first metal particles, all of said metal particles' being dispersed throughout said absorbent layer, said metal particles being spaced from 2.0 mm to touching and which due to their spacing either generate a sustained galvanic current of between 0.1 to 1.0 millivolts when subjected to an electrolyte-containing fluid or the metal particles act separately to kill pathogens.

11. A composition of matter in a form suitable for topical application to the epidermis or mucosa comprising at least one polymer and a plurality of first metal particles having an electrochemical potential, a plurality of second metal particles having a different electrochemical potential from said plurality of first metal particles, all of said metal particles' being dispersed throughout said composition of matter, said metal particles being spaced from between 0.1 mm to 7.0 mm apart or are touching and which due to their spacing generate a sustained galvanic current of between 0.1 to 1.0 millivolts when subjected to an electrolyte-containing fluid.

12. A composition of matter in a form suitable for topical application to the epidermis or mucosa comprising at least one polymer and a plurality of first metal particles having an electrochemical potential, a plurality of second metal particles having a different electrochemical potential from said plurality of first metal particles, all of said metal particles' being dispersed throughout said composition of matter, said metal particles being spaced from about 2 mm to touching.

13. The composition of matter according to claim 12 wherein said at least one polymer forms part of a lotion, cream or gel.

14. The composition of matter according to claim 12 wherein said at least one polymer forms a part of a polymeric construct of an earplug, a nose plug, a suppository, a rectal comfort pad, an adhesive heating pad or a joint pad.

15. The composition of matter according to claim 12 wherein said at least one polymer forms a part of a topical gel for epidermal or mucosal administration.

16. The composition of matter according to claim 15 wherein said gel is a gel for vaginal administration

17. The composition of matter according to claim 15 wherein said gel is a gel for nasal administration.

18. The composition of matter according to claim 16 wherein said gel contains particulate hydrated aluminum potassium sulfate dispersed throughout and wherein said first metal particles are silver.

19. The composition of matter according to claim 18 wherein said particulate hydrated aluminum potassium sulfate dispersed throughout said gel is present in the amount of about 1-15% by weight of the gel.

20. The composition of matter according to claim 19 wherein said particulate hydrated aluminum potassium sulfate is present in the amount of about 2-8% by weight of the gel.

21. A combination article of manufacture comprising a feminine hygiene article comprising at least one polymer and at least two types of metals in particulate form distributed throughout said polymer and a test stick having on its end a quantity of a pH indicator of pre-selected pH.
